SEQUENCE LISTING (17 sequences)

SEQ ID NO: 1 - protein, 7 aa, Artificial Sequence
  cyclic peptide targeting CXCR4
  acetylation (1)..(1): acetylation of N- terminal Arg
  disulfid (3)..(7): disulfid bond between D-Cys (3) and Pen (7)
  L-2-Naphthylalanine (5)..(5): Xaa L-2-Naphthylalanine
  Penicillamine (7)..(7): Xaa Penicillamine
RACRXHX

SEQ ID NO: 2 - protein, 7 aa, Artificial Sequence
  cyclic peptide targeting CXCR4
  disulfid (3)..(7): disulfid bond between D-Cys (3) and Pen (7)
  L-2-Naphthylalanine (5)..(5): Xaa L-2-Naphthylalanine
  Penicillamine (7)..(7): Xaa Penicillamine
RACRXHX

SEQ ID NO: 3 - protein, 7 aa, Artificial Sequence
  cyclic peptide targeting CXCR4
  disulfid (3)..(7): disulfid bond between D-Cys (3) and Cys (7)
RACRFFC

SEQ ID NO: 4 - protein, 7 aa, Artificial Sequence
  cyclic peptide targeting CXCR4
  disulfid (3)..(7): disulfid bond between D-Cys (3) and Pen (7)
  Penicillamine (7)..(7): Xaa Penicillamine
RACRFHX

SEQ ID NO: 5 - protein, 7 aa, Artificial Sequence
  cyclic peptide targeting CXCR4
  disulfid (3)..(7): disulfid bond between D-Cys (3) and Pen (7)
  Penicillamine (7)..(7): Xaa Penicillamine
RACRFFX

SEQ ID NO: 6 - protein, 7 aa, Artificial Sequence
  cyclic peptide targeting CXCR4
  disulfid (3)..(7): disulfid bond between D-Cys (3) and Pen (7)
  L-2-Naphthylalanine (5)..(5): Xaa L-2-Naphthylalanine
  Penicillamine (7)..(7): Xaa Penicillamine
RACRXFX

SEQ ID NO: 7 - protein, 7 aa, Artificial Sequence
  cyclic peptide targeting CXCR4
  disulfid (3)..(7): disulfid bond between D-Cys (3) and Cys (7)
  L-2-Naphthylalanine (5)..(5): Xaa L-2-Naphthylalanine
RACRXFC

SEQ ID NO: 8 - protein, 7 aa, Artificial Sequence
  cyclic peptide targeting CXCR4
  disulfid (3)..(7): disulfid bond between D-Cys (3) and Cys (7)
  L-2-Naphthylalanine (5)..(5): Xaa L-2-Naphthylalanine
RACRXHC

SEQ ID NO: 9 - protein, 7 aa, Artificial Sequence
  cyclic peptide targeting CXCR4
  disulfid (3)..(7): disulfid bond between D-Cys (3) and Cys (7)
RACRFHC

SEQ ID NO: 10 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Pen (7)
  L-2-Naphthylalanine (5)..(5): Xaa L-2-Naphthylalanine
  Penicillamine (7)..(7): Xaa Penicillamine
RACRXHX

SEQ ID NO: 11 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Cys (7)
RACRFFC

SEQ ID NO: 12 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Pen (7)
  Penicillamine (7)..(7): Xaa Penicillamine
RACRFHX

SEQ ID NO: 13 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Pen (7)
  Penicillamine (7)..(7): Xaa Penicillamine
RACRFFX

SEQ ID NO: 14 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Pen (7)
  L-2-Naphthylalanine (5)..(5): Xaa L-2-Naphthylalanine
  Penicillamine (7)..(7): Xaa Penicillamine
RACRXFX

SEQ ID NO: 15 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Cys (7)
  L-2-Naphthylalanine (5)..(5): Xaa L-2-Naphthylalanine
RACRXFC

SEQ ID NO: 16 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Cys (7)
  L-2-Naphthylalanine (5)..(5): Xaa L-2-Naphthylalanine
RACRXHC

SEQ ID NO: 17 - protein, 7 aa, Artificial Sequence
  cyclic peptide targeting CXCR4
  acylation (1)..(1): CH3(CH2)nCO bound to N- terminal Arg, wherein n is from 0 to 30
  disulfid (3)..(7): disulfid bond between D-Cys (3) and Cys (7)
RACRFHC

(19) United States
(12) Patent Application Publication
(10) Pub. No.: US 2023/0099200 A1
(43) Pub. Date: Mar. 30, 2023

(54) RADIOLABELED PEPTIDES FOR NON-INVASIVE DIAGNOSIS AND TREATMENT OF CXCR4 EXPRESSING TUMORS

(71) Applicant: ISTITUTO NAZIONALE TUMORI IRCCS - FONDAZIONE G. PASCALE, Napoli (NA) (IT)

(72) Inventors: Stefania SCALA, Napoli (NA) (IT); Anna Maria TROTTA, Napoli (NA) (IT)

(73) Assignee: ISTITUTO NAZIONALE TUMORI IRCCS - FONDAZIONE G. PASCALE, Napoli (NA) (IT)

(21) Appl. No.: 17/789,398

(22) PCT Filed: Dec. 23, 2020
(86) PCT No.: PCT/EP2020/087792

§ 371 Date: Jun. 27, 2022

(30) Foreign Application Priority Data
Dec. 27, 2019 (IT) 102019000025588

(51) Int. Cl. A61K 51/08; A61K 45/06; A61P 35/00

(52) U.S. Cl. CPC A61K 51/088; A61K 45/06; A61P 35/00

(57) ABSTRACT

The present invention concerns radiolabeled peptides that are suitable tracers for specific targeting and imaging of human CXCR4 in vivo, in particular for the detection of human primary and secondary CXCR4 overexpressing tumors. In addition, the radiolabeled peptides according to the present invention can be advantageously used in the treatment of human primary and secondary CXCR4 overexpressing tumors.

The present invention concerns radiolabeled peptides for non-invasive diagnosis and treatment of CXCR4 expressing tumors.

In particular, the present invention concerns radiolabeled peptides that are suitable tracers for specific targeting and imaging of human CXCR4 expressing cells, for example in primary and secondary tumors, neoplastic and tumor infiltrating immune cells. In addition, the radiolabeled peptides, according to the present invention, can be advantageously used in the treatment of CXCR4 expressing disorders, such as the above described tumors.

The chemokine receptor 4 (CXCR4) is an evolutionarily highly conserved G-protein coupled receptor (GPCR) which is physiologically expressed on monocytes, B cells, naive T cells, neutrophils and eosinophils. CXCR4 is also overexpressed in more than 30 different human cancers where it promotes tumor growth, invasion, angiogenesis and metastasis [1-4]. High CXCR4 expression has been reported in numerous solid cancers and increased CXCR4 expression is associated with an aggressive phenotype [5-7].

In this context, different strategies aiming at CXCR4 inhibition have been pursued, leading to the development of potent low molecular weight CXCR4 antagonists. By interfering with the CXCR4-CXCL12 axis, these compounds strongly promote white blood cell mobilization and prevent distant metastasis by impairing migration and homing of cancer cells. Several CXCR4 antagonists have been reported, including antibodies [8], small-molecules [9-11] and peptides [12-14]. Amongst them, the bicyclam AMD3100 (plerixafor/Mozobil) is the only FDA approved CXCR4-targeted compound, which is indicated for the mobilization of hematological stem cells [15].

CXCR4 antagonists have been shown to modify the tumor microenvironment (TME) potentiating tumoral T cell access [16-18]; based on this finding, combination therapies of CXCR4 antagonists with immune checkpoints inhibitors targeting PD-1 or PD-L1 are currently under evaluation [(NCT02472977),(NCT02737072), (NCT02823405), (NCT02923531)].

A variety of CXCR4-targeted imaging agents for positron emission tomography (PET) have been developed, ranging from radiolabeled AMD3100-based analogs over peptidic radiopharmaceuticals to full-size antibodies. From all three classes, highly promising candidates with high CXCR4 affinity and excellent CXCR4 targeting properties have emerged from preclinical studies and single representatives such as [⁶⁴Cu]AMD3100 [19], the T-140 analog [⁶⁸Ga]NOTA-NFB [20] and the cyclic pentapeptide [⁶⁸Ga]pentixafor [21-22] have been evaluated in patients. Unfortunately, the clearance pattern of the first two compounds ([⁶⁴Cu]AMD3100 and [⁶⁸Ga]NOTA-NFB), both of which exhibit considerable to very high uptake in mice and human liver and spleen, challenge their applicability. [⁶⁸Ga]Pentixafor targeting CXCR4 has successfully been evaluated preclinically and clinically in different malignancies [23-29]. However, uptake of [⁶⁸Ga]Pentixafor in solid tumors was lower as compared to [¹⁸F]FDG PET[23].

In the light of the above, there is a need for new CXCR4-targeting agents for positron emission tomography (PET) and therapy able to overcome the disadvantages of known agents. In particular, there is a need for CXCR4-targeted PET probes with improved affinity, which may provide improved sensitivity in the detection of CXCR4 overexpressing disorders, such as human primary and secondary CXCR4 overexpressing solid tumors. A new family of CXCR4 peptide antagonists was recently developed through rational design and iterative optimizations (WO2011092575), leading to the highly specific CXCR4 antagonist named R54 [30-32], having sequence (Ac-Arg-Ala-[DCys-Arg-Nal(2′)-His-Pen]COOH) (SEQ ID NO:1).

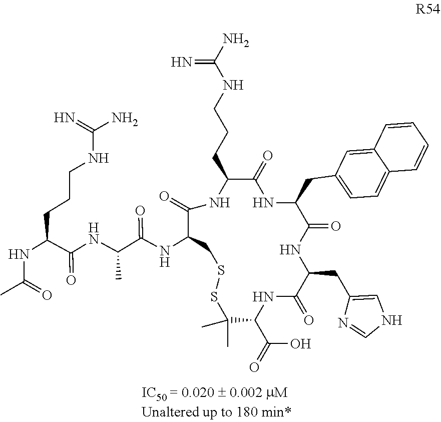 [SMILES: CC(=O)N[C@@H](CCCNC(=N)N)C(=O)N[C@@H](C)C(=O)N[C@@H]1CSSC(C)(C)[C@@H](C(=O)O)NC(=O)[C@H](Cc2c[nH]cn2)NC(=O)[C@H](Cc2ccc3ccccc3c2)NC(=O)[C@H](CCCNC(=N)N)NC1=O]

R54 is a low molecular weight cyclic peptide consisting of seven amino acids with a disulfide bridge between D-Cys3 and Pen7. This compound showed high serum stability, high CXCR4 affinity (IC₅₀=1.5±0.5 nM in competitive binding assay using anti-CXCR4 mAb 12G5 and IC₅₀=20±2 nM extrapolated by competition binding assays with ¹²⁵I-CXCL12) and potent antagonist effect [32].

According to the present invention labeled R54 and similar peptides have now been synthetized that efficiently target CXCR4 expressing cells, such as CXCR4 expressing tumor cells. Therefore, labeled peptide of the present invention can be advantageously used for PET non-invasive imaging and for the treatment of CXCR4 expressing disorders, such as CXCR4 expressing tumors.

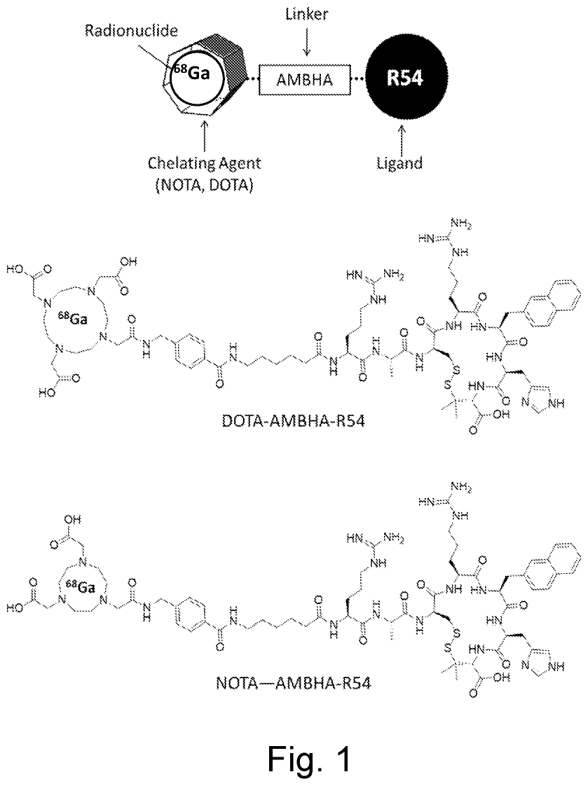

In particular, as described below in the examples the N-terminus of R54 was selected as position to introduce 6-(4-(aminomethyl)benzamido)hexanoic acid as linker (AMBHA), which in turn was functionalized with 1,4,7-triazacyclononane-triacetic acid (NOTA) and 1,4,7,10-tetraazacyclododecane-1,4,7,10-tetraacetic acid (DOTA) as metal chelators (FIG. 1).

The AMBHA choice takes into account the adequate distance between the metal chelator moiety and the receptor binding site provided by the combination of 6-aminohexanoic acid (Ahx) and 4-(aminomethyl)benzoic acid (AMBA) provide thus minimizing the influence of chelator conjugation on CXCR4 affinity.

The resulting NOTA-AMBHA-R54 and DOTA-AMBHA-R54 were labelled with ⁶⁸Ga and engaged in vitro and in vivo characterization study, demonstrating their suitability for high-contrast CXCR4 PET imaging.

Specifically, according to the present invention, DOTA and NOTA-AMBHA-R54 were synthesized by combining solid and solution-phase peptide synthesis. Binding affinities (IC₅₀) were determined using CXCR4-expressing CCRF-CEM cells, human T lymphoblastic leukemia cells CXCR4 expressing, with [¹²⁵I]CXCL12 as radioligand. Chinese hamster ovary (CHO) and CHO cells transduced with human CXCR4 (CHO-hCXCR4) grown in complete medium supplemented with 1 mg/mL G418 (a kind gift of Dr. David McDermott (NIAID, NIH, Bethesda, Md.).

PET imaging and biodistribution studies were carried out in athymic mice bearing subcutaneous human CXCR4 (CHO-hCXCR4)tumors. CXCR4 expression on CHO-hCXCR4 and CHO tumors sections was confirmed using immunohistochemistry.

The results obtained according to the present invention show that compared to parent peptide R54, NOTA-AMBHA-R54 displays slightly decreased CXCR4 affinity, whereas DOTA conjugation clearly reduced CXCR4 affinity (Table 1).

[⁶⁸Ga]NOTA-AMBHA-R54 efficiently and specifically accumulated in CXCR4-expressing -tumors as compared to its DOTA-counterpart in CHO-hCXCR4 bearing athymic mice (4.44±0.84 vs 1.63±0.84% ID/g at 45 min post injection).

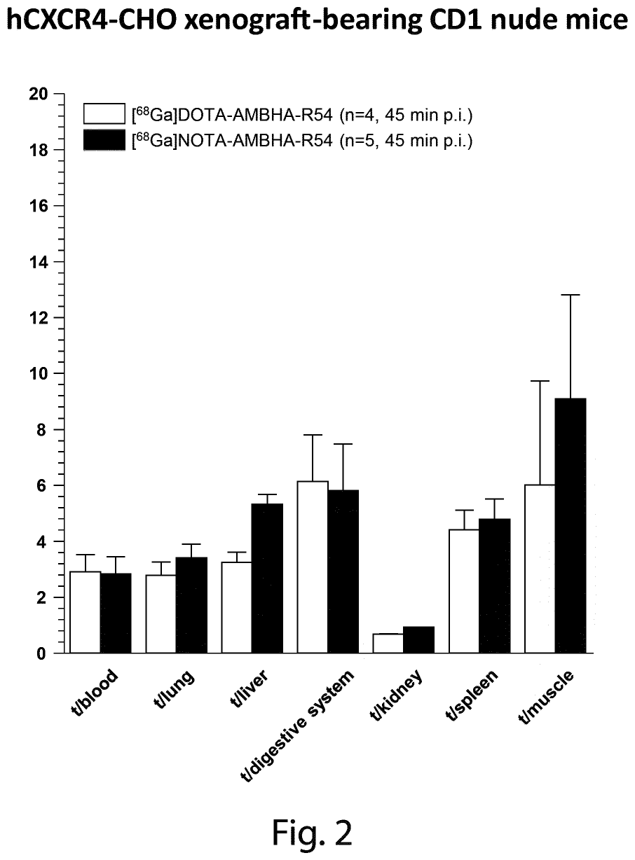

The tumor accumulation of [⁶⁸Ga]DOTA- and [⁶⁸Ga]NOTA-AMBHA-R54 in CHO-CXCR4 xenografts, alongside with their efficient background clearance, led to high tumor/non-tumor ratios (except for kidney) for both analogs (FIG. 2), with values for [⁶⁸Ga]NOTA-AMBHA-R54 being substantially higher than for the DOTA analog. In addition [⁶⁸Ga]NOTA-AMBHA-R54 shows high background clearance virtually no uptake in the hepatobiliary system and exclusive renal excretion. Thus [⁶⁸Ga]NOTA-AMBHA-R54 is expected to allow sensitive detection of abdominal lesions.

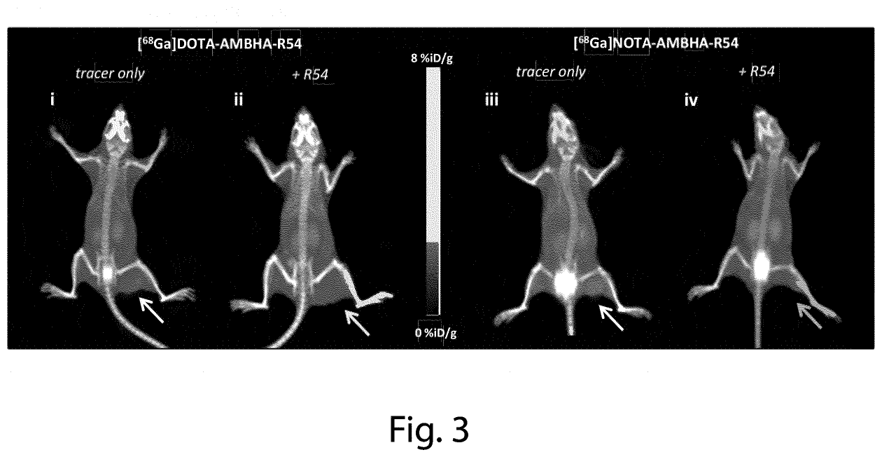

PET imaging studies demonstrated efficient CXCR4 targeting by [⁶⁸Ga]DOTA-AMBHA-R54 and [⁶⁸Ga]NOTA-AMBHA-R54, with the NOTA-analog showing the anticipated superior performance, including both more efficient background clearance and higher accumulation in the CHO-hCXCR4 (FIG. 3iii), compared to the DOTA-peptide.

Interestingly, in CHO-hCXCR4 xenografts, the differences in maximum standardized uptake value (SUVmax) obtained for [⁶⁸Ga]NOTA-AMBHA-R54 and [⁶⁸Ga]DOTA-AMBHA-R54 (19.6±2.00 vs 2.02±0.14 g/mL respectively, p=0.05) were even higher than anticipated from the biodistribution experiments. Moreover, when the tracer injection is concomitant with injection of excess of unlabeled R54, tracer accumulation is significantly reduced in CXCR4-expressing tumor, indicating a specific competition of unlabelled R54 with [⁶⁸Ga]NOTA/DOTA-Ahx-R54 tracer for CXCR4 expressing cells.

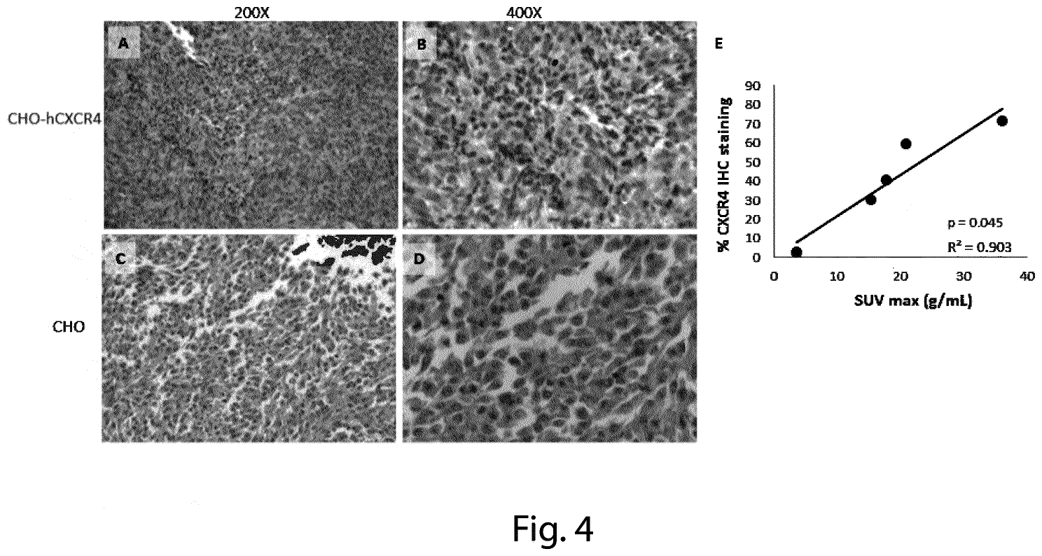

To correlate [⁶⁸Ga]NOTA-AMBHA-R54 uptake with the CXCR4 expression in tumor tissue, CXCR4 immunohistochemical staining was performed in CHO-hCXCR4 and CHO tumors. In FIG. 4A the mean percentage of CXCR4 positive cells was 56.6 for CHO-hCXCR4 vs 16.05 for CHO tumors, respectively. The Spearman test revealed a strong correlation between SUVmax of [⁶⁸Ga]NOTA-AMBHA-R54 PET and the CXCR4 expression in the relative tumor samples (R2=0.903, p=0.045, FIG. 4).

Therefore, the results according to the present invention show that the labeled peptides[⁶⁸Ga]NOTA-AMBHA-R54 and [⁶⁸Ga]DOTA-AMBHA-R54 can advantageously be used as PET tracers for imaging of CXCR4 expressing cells, such as in primary and secondary CXCR4 overexpressing tumors. Moreover, given the high affinity and selectivity for human CXCR4, rapid renal excretion and very low non-specific background accumulation, a suitable therapeutic counterpart based on the NOTA-AMBHA-R54 scaffold as a targeting vector will be evaluated.

It is therefore specific object of the present invention a radiopharmaceutical compound targeting CXCR4 receptor, said compound comprising or consisting of:

a) a cyclic peptide, as monomer or multimer, having the following formula I:

Arg-Ala-[D-Cys-Arg-X-Y-Z]—COOH

wherein

X is L-2-Nal or L-Phe, preferably L-2-Nal;

Y is L-Phe or L-His, preferably L-His;

Z is L-Cys or L-Pen, preferably L-Pen;

b) a linker, said linker being bound to the N-terminus of said cyclic peptide;

c) a chelator, said chelator being bound to said linker; and

d) a radioisotope, said radioisotope being bound to said chelator. The cyclic peptide has a disulfide bridge between D-Cys3 and Z.

In particular, the cyclic peptide of the radiopharmaceutical compound according to the present invention is chosen among the following cyclic peptides:

(SEQ ID NO: 2)
Arg-Ala-[D-Cys-Arg-2-Nal-His-Pen]-COOH
(SEQ ID NO: 3)
Arg-Ala-[D-Cys-Arg-Phe-Phe-Cys]-COOH
(SEQ ID NO: 4)
Arg-Ala-[D-Cys-Arg-Phe-His-Pen]-COOH
(SEQ ID NO: 5)
Arg-Ala- [D-Cys-Arg-Phe-Phe-Pen]-COOH
(SEQ ID NO: 6)
Arg-Ala-[D-Cys-Arg-2-Nal-Phe-Pen]-COOH
(SEQ ID NO: 7)
Arg-Ala-[D-Cys-Arg-2-Nal-Phe-Cys]-COOH
(SEQ ID NO: 8)
Arg-Ala-[D-Cys-Arg-2-Nal-His-Cys]-COOH
(SEQ ID NO: 9)
Arg-Ala-[D-Cys-Arg-Phe-His-Cys]-COOH;

preferably SEQ ID NO:2, SEQ ID NO:7, SEQ ID NO:8, SEQ ID NO:9.

According to the present invention, the linker can be a combination of at least two subunits A and B, wherein A is bound to said chelator, whereas B is bound to the N-terminus of said cyclic peptide, subunits A and B being connected each other by amide bond or by a subunit C, preferably by amide bond, wherein

subunit A can be chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected each other by amide bond; or (4-Ethynylphenyl)methanamine, (3-Ethynylphenyl)methanamine, 3-Ethynylaniline, 4-Ethynylaniline, 4-Pentyn-1-amine, But-3-yn-1-amine, propargylamine, 3-Azido-1-propanamine, 4-azido-1-butylamine, 5-azido-1-pentylamine, 6-azido-1-hexylamine, when subunits A and B are connected each other by a subunit C; preferably 4-aminomethylbenzoic acid;

subunit B can be chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected each other by amide bond; or

2-azidobenzoic acid, 3-azidobenzoic acid, 4-azidobenzoic acid, 2-azidomethyl benzoic acid, 3-azidomethylbenzoic acid; 4-azidomethylbenzoic acid, Azido-(PEG)n-COOH, wherein n is from 2 to 10, 8-azidooctanoic acid, 7-azidoheptanoic acid, 6-azidohexanoic acid, 5-azidovaleric acid, 4-azidobutyric acid, 3-butynoic acid, 4-pentynoic acid and 6-hexynoic acid, when subunits A and B are connected each other by a subunit C; preferably 6-aminohexanoic acid; and

subunit C can be chosen from the group consisting of 1,4 substituted 1,2,3 triazole or 1,5 substituted 1,2,3 triazole linkers. Depending on the subunits A and B employed to build the linker, 1,4 and 1,5 substituents are aliphatic and/or aromatic moieties of which one is endowed with a carboxylic acid terminus to bind the N-terminus of the cyclic peptide and an amino terminus to bind to the said chelator.

According to an embodiment of the present invention, the chelator can be chosen from the group consisting of 1,4,7-triazacyclononane-triacetic acid (NOTA), 1,4,7,10-tetraazacyclododecane-1,4,7,10-tetraacetic acid (DOTA), diethylenetriaminepentaacetic acid (DTPA), {4-[2-(bis-carboxymethylamino)-ethyl]-7-carboxymethyl-[1,4,7]triazonan-1-yl}-acetic acid (NETA), 1,4,8,11-tetraazacyclotetradecane1,4,8,11-tetraacetic acid (TETA), p-SCN-Bn-NOTA(C-NOTA), NODASA, NODAGA, C-DOTA, DOTAGA, TRAP(Azide)₁, TRAP(Azide)₂, TRAP(Azide)₃ (NOTA derivatives for the preparation of monomers, multimers, for example dimers or trimers); preferably 1,4,7-triazacyclononane-triacetic acid (NOTA), 1,4,7,10-tetraazacyclododecane-1,4,7,10-tetraacetic acid (DOTA), more preferably 1,4,7-triazacyclononane-triacetic acid (NOTA).

According to the present invention, the radioisotope can be chosen from the group consisting of ⁶⁸Ga³⁺, ⁶⁷Ga³⁺, ⁶⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺, ²¹²Pb²⁺ or ¹⁸F⁻, wherein

⁶⁸Ga³⁺, ⁶⁷Ga³⁺, ⁶⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺ or ²¹²Pb²⁺ is suitable preferably for DOTA, p-SCN-Bn-NOTA(C-NOTA), NODASA, NODAGA, C-DOTA and DOTAGA; ⁸⁸Ga³⁺, ⁸⁷Ga³⁺, ⁸⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺ or ¹⁸F⁻ is suitable preferably for NOTA, DTPA, NETA, TETA;

⁶⁸Ga³⁺, ⁶⁷Ga³⁺ are suitable for TRAP (Azide)₁, TRAP(Azide)₂, TRAP(Azide)₃ (NOTA derivatives for the preparation of monomers, dimers e trimers); preferably ⁸⁸Ga³⁺.

The present invention concerns also a precursor compound of the radiopharmaceutical compound as defined above, said precursor comprising or consisting of a

a cyclic peptide, as monomer or multimer, having the following formula I:

Arg-Ala-[D-Cys-Arg-X-Y-Z]—COOH

wherein

X is L-2-Nal or L-Phe, preferably L-2-Nal;

Y is L-Phe or L-His, preferably L-His;

Z is L-Cys or L-Pen, preferably L-Pen.

b) a linker, said linker being bound to the N-terminus of said cyclic peptide;

c) a chelator, said chelator being bound to said linker.

In particular, the cyclic peptide of the precursor compound according to the present invention is chosen among the following cyclic peptides:

(SEQ ID NO: 2)
Arg-Ala-[D-Cys-Arg-2-Nal-His-Pen]-COOH
(SEQ ID NO: 3)
Arg-Ala-[D-Cys-Arg-Phe-Phe-Cys]-COOH
(SEQ ID NO: 4)
Arg-Ala-[D-Cys-Arg-Phe-His-Pen]-COOH
(SEQ ID NO: 5)
Arg-Ala-[D-Cys-Arg-Phe-Phe-Pen]-COOH
(SEQ ID NO: 6)
Arg-Ala-[D-Cys-Arg-2-Nal-Phe-Pen]-COOH
(SEQ ID NO: 7)
Arg-Ala-[D-Cys-Arg-2-Nal-Phe-Cys]-COOH
(SEQ ID NO: 8)
Arg-Ala-[D-Cys-Arg-2-Nal-His-Cys]-COOH
(SEQ ID NO: 9)
Arg-Ala-[D-Cys-Arg-Phe-His-Cys]-COOH;

preferably SEQ ID NO:2, SEQ ID NO:7, SEQ ID NO:8, SEQ ID NO:9.

As mentioned above, the linker can be a combination of at least two subunits A and B, wherein A is bound to said chelator, whereas B is bound to the N-terminus of said cyclic peptide, subunits A and B being connected each other by amide bond or by a subunit C, preferably by amide bond,

wherein

subunit A can be chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected each other by amide bond; or

(4-Ethynylphenyl)methanamine, (3-Ethynylphenyl)methanamine, 3-Ethynylaniline, 4-Ethynylaniline, 4-Pentyn-1-amine, But-3-yn amine, propargylamine, 3-Azido-1-propanamine, 4-azido butylamine, 5-azido-1-pentylamine, 6-azido-1-hexylamine, when subunits A and B are connected each other by a subunit C, preferably 4-aminomethylbenzoic acid;

subunit B can be chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected each other by amide bond; or

2-azidobenzoic acid, 3-azidobenzoic acid, 4-azidobenzoic acid, 2-azidomethyl benzoic acid, 3-azidomethylbenzoic acid; 4-azidomethylbenzoic acid, Azido-(PEG)n-COOH wherein n is from 2 to 10, 8-azidooctanoic acid, 7-azidoheptanoic acid, 6-azidohexanoic acid, 5-azidovaleric acid, 4-azidobutyric acid, 3-butynoic acid, 4-pentynoic acid and 6-hexynoic acid, when subunits A and B are connected each other by a subunit C; preferably 6-aminohexanoic acid; and

subunit C can be chosen from the group consisting of 1,4 substituted 1,2,3 triazole or 1,5 substituted 1,2,3 triazole linkers. Depending on the subunits A and B employed to build the linker, 1,4 and 1,5 substituents are aliphatic and/or aromatic moieties of which one is endowed with a carboxylic acid terminus to bind the N-terminus of the cyclic peptide and an amino terminus to bind to the said chelator.

According to the present invention, the chelator can be chosen from the group consisting of 1,4,7-triazacyclononane-triacetic acid (NOTA), 1,4,7,10-tetraazacyclododecane-1,4,7,10-tetraacetic acid (DOTA), diethylenetriaminepentaacetic acid (DTPA), {4-[2-(bis-carboxymethylamino)-ethyl]-7-carboxymethyl-[1,4,7]triazonan-1-yl}-acetic acid (NETA), 1,4,8,11-tetraazacyclotetradecane1,4,8,11-tetraacetic acid (TETA), p-SCN-Bn-NOTA(C-NOTA), NODASA, NODAGA, C-DOTA, DOTAGA, TRAP(Azide)₁, TRAP(Azide)₂, TRAP(Azide)₂ (NOTA derivatives for the preparation of monomers, multimers, for example dimers or trimers); preferably 1,4,7-triazacyclononane-triacetic acid (NOTA), 1,4,7,10-tetraazacyclododecane-1,4,7,10-tetraacetic acid (DOTA), more preferably 1,4,7-triazacyclononane-triacetic acid (NOTA).

The present invention concerns also a radiopharmaceutical composition comprising or consisting of the radiopharmaceutical compound as defined above, in association with one or more excipients and/or adjuvants.

In addition the present invention concerns a pharmaceutical composition comprising or consisting of the precursor compound as defined above, in association with one or more excipients and/or adjuvants.

The present invention concerns also the radiopharmaceutical compound as defined above, the precursor compound as defined above, the radiopharmaceutical or pharmaceutical composition as defined above for use in medical field.

In particular, the present invention concerns the radiopharmaceutical compound or the radiopharmaceutical composition as defined above for use in in vivo diagnostic methods for locating or imaging CXCR4 receptor positive cells, such as CXCR4 expressing tumour cells such as by PET imaging diagnostic method.

A further object of the present invention is the use of the radiopharmaceutical compound as defined above or the radiopharmaceutical composition as defined above as imaging agent in PET imaging.

In addition, the present invention concerns the radiopharmaceutical compound or the radiopharmaceutical composition as defined above for use in the treatment of CXCR4 expressing disorders, such as CXCR4 expressing tumours. Preferably, when the radiopharmaceutical compound or the radiopharmaceutical composition is used in therapy, the radioisotope can be chosen from the group of ⁶⁴Cu²⁺, ⁴⁴Sc³⁺, ⁴⁷Sc³⁺, ¹⁷⁷Lu³⁺, ⁸⁶Y³⁺, ⁹⁰Y³⁺, or ²¹³Bi³⁺ that are therapeutic isotopes.

In particular, the present invention can be applied to solid tumors such as Non-small-cell lung carcinoma, pancreatic cancer, prostate cancer, breast cancer, glioblastoma, renal cancer, sarcoma, colon cancer, melanoma, lung cancer, neuroendocrine tumors.

A further object of the present invention is a method for obtaining a radiopharmaceutical compound as defined above or a radiopharmaceutical composition as defined above, said method comprising:

a) radiolabeling a precursor compound as defined above or a pharmaceutical composition as defined above with a radioisotope chosen from the group consisting of ⁶⁸Ga³⁺, ⁶⁷Ga³⁺, ⁶⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺, ²¹²Pb²⁺ or ¹⁸F⁻, wherein

⁶⁸Ga³⁺, ⁶⁷Ga³⁺, ⁶⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺, ²¹²Pb²⁺ is suitable preferably for DOTA, p-SCN-Bn-NOTA(C-NOTA), NODASA, NODAGA, C-DOTA and DOTAGA;

⁶⁸Ga³⁺, ⁶⁷Ga³⁺, ⁶⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺, ²¹²Pb²⁺ or ¹⁸F⁻, is suitable preferably for NOTA, DTPA, NETA, TETA;

⁶⁸Ga³⁺, ⁶⁷Ga³⁺, are suitable for TRAP(Azide)₁, TRAP (Azide)₂, TRAP(Azide)₃ (NOTA derivatives for the preparation of monomers, dimers e trimers); preferably ⁶⁸Ga³⁺.

The present invention concerns also a kit for the preparation of a radiopharmaceutical compound as defined above or a radiopharmaceutical composition as defined above, said kit comprising or consisting of:

a precursor compound as defined above or a pharmaceutical composition as defined above.

A further object of the present invention is also a cyclic peptide, monomer or multimer, or a pharmaceutical composition comprising said cyclic peptide, for use in the treatment of CXCR4 expressing disorders, such as CXCR4 expressing tumours, wherein said cyclic peptide has the following formula:

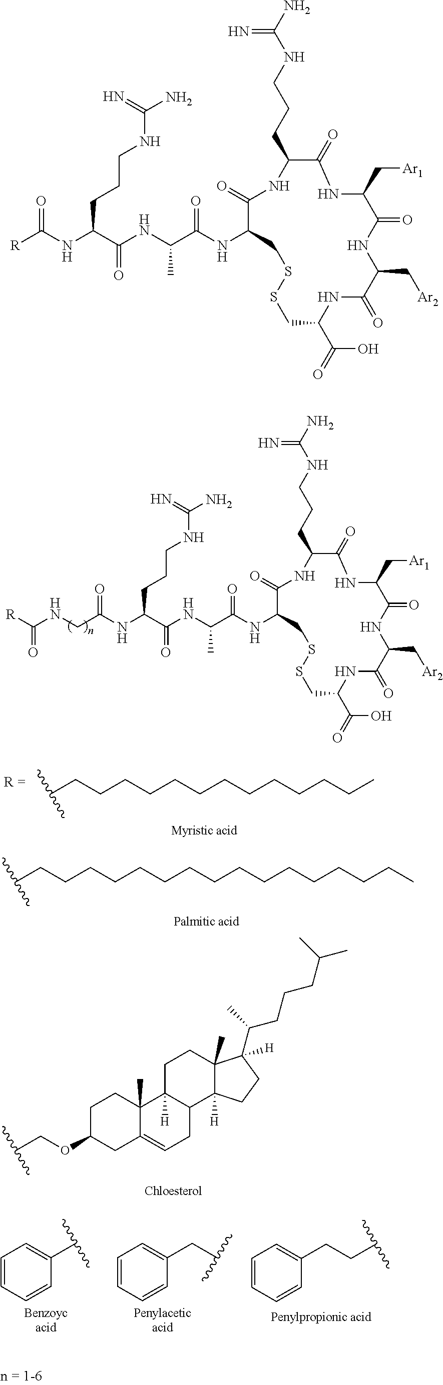

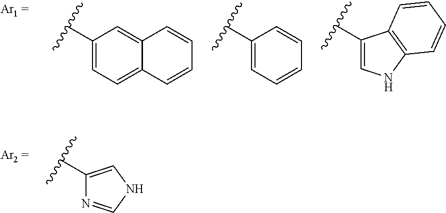

The synthesis of the lipidated peptidomimetic is carried out applying an ultrasonic-assisted Fmoc/tBu solid phase peptide synthesis (US-SPPS) previously described [33].

After elongation of linear amino acidic sequence, lipidic moieties selected is introduced at the N-terminal region of the resin bound peptide by means of an amide bond. This strategy allows to functionalize the peptides, avoiding additional steps in solution that might affect the synthetic efficiency. Once functionalized, the lipidated peptidomimetic is cleaved from the resin by acid treatment and the disulfide bond is formed by treating with N-chlorosuccinimide. Finally, the crude mixture is purified by reverse-phase preparative HPLC and the product characterized by analytical HPLC and MS-ESI spectrometry. The incorporation of lipid units onto a peptide backbone dramatically increases enzymatic stability (Simerska et al. 2011), receptor selectivity and potency (Ward et al. 2013), bioavailability (Hamman et al. 2005; Park et al. 2011; Renukuntla et al. 2013; Karsdal et al. 2015) and drug delivery potential (membrane permeability) (Zhang and Bulaj 2012; Simerska et al. 2011).Peptide lipidation has been exploited as effective strategy to improve both the pharmacokinetic and pharmacodynamic properties of the cyclic heptapeptides.

Preferably the CXCR4 expressing tumours are solid tumors such as Non-small-cell lung carcinoma, pancreatic cancer, prostate cancer, breast cancer, glioblastoma, sarcoma, colon cancer, melanoma, lung cancer, neuroendocrine tumors, renal cancer, preferably CXCR4 expression is on tumoral cells expressing the receptor and/or on tumor microenvironment cells such as T-cells, Macrophages, Myeloid Cells, Granulocytes and Endhotelial cells. Preferably said peptides is used in association with an immunomodulating therapy such as immunecheckpoints inhibitory antibodies (anti-CTLA4, anti-PD-1, anti-PD-L1).

The peptides of the invention display in vitro CXCR4 powerful inhibitory activity (Binding affinity I.C.50 1.5-53 nM).

According to the present invention, CXCR4 expressing tumours can also be, for example, solid tumors such as Non-small-cell lung carcinoma, pancreatic cancer, prostate cancer, breast cancer, glioblastoma, sarcoma, colon cancer, melanoma, lung cancer, neuroendocrine tumors, renal cancer.

The invention further provides a cyclic peptide or a pharmaceutical composition comprising said cyclic peptide, said cyclic peptide having formula II:

W-Arg-Ala-[D-Cys-Arg-X-Y-Z]—COOH

wherein

X is L-2-Nal or L-Phe, preferably L-2-Nal;

Y is L-Phe or L-His, preferably L-His;

Z is L-Cys or L-Pen, preferably L-Pen,

W is CH₃— (CH₂)ₙ—CO,

wherein n is from 1 to 30 when said cyclic peptide is chosen from the group consisting of:

(SEQ ID NO: 10)
CH₃(CH₂)ₙCO-Arg-Ala-[D-Cys-Arg-2-Nal-His-Pen]-COOH
(SEQ ID NO: 11)
CH₃(CH₂)ₙCO-Arg-Ala-[D-Cys-Arg-Phe-Phe-Cys]-COOH
(SEQ ID NO: 12)
CH₃(CH₂)ₙCO-Arg-Ala-[D-Cys-Arg-Phe-His-Pen]-COOH
(SEQ ID NO: 13)
CH₃(CH₂)ₙCO-Arg-Ala-[D-Cys-Arg-Phe-Phe-Pen]-COOH
(SEQ ID NO: 14)
CH₃(CH₂)ₙCO-Arg-Ala-[D-Cys-Arg-2-Nal-Phe-Pen]-
COOH;

and,

wherein n is from 0 to 30 when said cyclic peptide is chosen from the group consisting of:

(SEQ ID NO: 15)
CH₃(CH₂)ₙCO-Arg-Ala-[D-Cys-Arg-2-Nal-Phe-Cys]-COOH
(SEQ ID NO: 16)
CH₃(CH₂)ₙCO-Arg-Ala-[D-Cys-Arg-2-Nal-His-Cys]-COOH
(SEQ ID NO: 17)
CH₃(CH₂)ₙCO-Arg-Ala-[D-Cys-Arg-Phe-His-Cys]-COOH.

Such peptides have advantageous properties. Preferably the cyclic peptide or the pharmaceutical composition comprising said cyclic peptide is for use in the treatment of CXCR4 expressing disorders, such as CXCR4 expressing tumours, preferably the CXCR4 expressing tumours are solid tumors such as Non-small-cell lung carcinoma, pancreatic cancer, prostate cancer, breast cancer, glioblastoma, sarcoma, colon cancer, melanoma, lung cancer, neuroendocrine tumors, renal cancer, preferably CXCR4 expression is on tumoral cells expressing the receptor and/or on tumor microenvironment cells such as T-cells, Macrophages, Myeloid Cells, Granulocytes and Endhotelial cells. Still preferably said peptides is used in association with an immunomodulating therapy.

The peptides used in the present invention comprise both monomeric, dimeric or multimeric cyclic peptides, their combinations and pharmacologically acceptable salts.

The present invention now will be described by an illustrative, but not limitative way, according to preferred embodiments thereof, with particular reference to the enclosed drawings, wherein:

FIG. 1 shows a schematic representation of the R54-peptide based PET probes. The linker (AMBHA) and a metal chelator (DOTA or NOTA) were conjugated at the amino terminus of R54 peptide. AMBHA: 6-(4-(aminomethyl)benzamido)hexanoic acid; DOTA: 1,4,7,10-tetraazacyclododecane-1,4,7,10-tetraacetic acid; NOTA: 1,4,7-triazacyclononane-triacetic acid.

FIG. 2 shows tumor-to-organ ratios values of [⁶⁸Ga]DOTA-AMBHA-R54 (white bars) and [⁶⁸Ga]NOTA-AMBHA-R54 (black bars) in h-CXCR4-CHO xenograft-bearing CD1 nude mice. Data are expressed as mean SD (n 4-6).

FIG. 3 shows the representative whole-body coronal PET/CT images (MIP) of [⁶⁸Ga]DOTA- and [⁶⁸Ga]NOTA-AMBHA-R54 in CHO-hCXCR4 (right flank) bearing CD1 mice at 45-60 min p.i. (i-iii) tracer only, (ii-iv) or coinjection of 8 mg/kg of peptide R54. Arrows indicate the position of the tumor.

FIG. 4 shows the correlation of [⁶⁸Ga]NOTA-AMBHA-R54 uptake (SUVmax) and CXCR4 expression assessed by IHC. Representative H&E staining and CXCR4 immunohistochemistry (clone mab172) of (A-B) excised CHO-hCXCR4 and (C-D) CHO tumors at different magnification (A-C 200X; B-D 400X) confirming higher CXCR4 expression in CHO-hCXCR4 tumors. (E) The Spearman's rho correlation coefficients with P=0,0455 was used to measure the degree of association between SUV(max) and % CXCR4 IHC staining.

EXAMPLE 1: Synthesis of the radiolabeled peptides of the present invention and study concerning their CXCR4 affinities, in vivo biodistribution, in vivo CXCR4 PET imaging and uptake in CXCR4 expressing tumors

### MATERIALS AND METHODS

Synthesis of NOTA-AMBHA-R54, DOTA-AMBHA-R54, [^{nat}Ga]NOTA-and [^{nat}ga]DOTA-AMBRA-R54

The synthesis of the peptides was accomplished using 2-C1-TrtCl resin by applying an ultrasonic-assisted Fmoc/tBu solid phase peptide synthesis (US-SPPS) previously described [33].

After elongation of linear sequence, the resulting peptidomimetic and acidic sensitive protective groups were simultaneously cleaved from the resin treating with a solution of TFA/TIS for 2 h at room temperature. Oxidation of sulfide side chains was achieved dissolving the crude peptide in H₂O and adding an aqueous solution of N-chlorosuccinimide. The crude mixture was purified by reverse-phase preparative HPLC and the product was characterized by analytical HPLC and MS-ESI spectrometry.

A detailed description of synthesis, yield, purity, retention times, analytical data is provided below: Synthesis of NOTA-AMBHA-R54 and DOTA-AMBHA-R54

Standard Na-Fmoc-protected amino acids, O-benzotriazole-N, N, N′, N′-tetra-methyl-uroniumhexafluorophosphate (HBTU, purity 99%), N,N-diisopropylethylamine (DIEA, purity 99%), trifluoroacetic acid (TFA, purity 99%), piperidine, Fmoc-D-Cys(Trt)-OH, Fmoc-L-2Nal-OH, Fmoc-L-Pen(Trt)-OH, 4-(Fmoc-aminomethyl)benzoic acid and Fmoc-6-Ahx-OH were purchased from IRIS Biotech (Marktredwitz, Germany). DOTA-tris(tBu)ester and NOTA-bis(tBu)ester were purchased from CheMatech (Dijon, France). Triisopropylsilane (TIS) (purity 98%), 1-hydroxybenzotriazole hydrate (HOBt) (purity >97% dry weight, water ≈12%) (1H-7-Azabenzotriazol-1-yl-oxy)tris-pyrrolidinophosphonium hexafluorophosphate (PyAOP) (purity 96%) %), 1-hydroxy-7-azabenzotriazole (HOAt, purity 96%), N-chloro succinimide (purity 98%), acetic anhydride (Ac20, purity >98%) %), anhydrous N,N-dimethylformamide (DMF), and anhydrous dichloromethane (DCM) were purchased from Sigma-Aldrich (Milano, Italy).

Peptide synthesis solvents, water and acetonitrile for HPLC, were reagent grade and were acquired from commercial sources (Sigma-Aldrich, Milano, Italy) and used without any further purification unless otherwise stated. Peptides were purified by preparative HPLC (Shimadzu HPLC system) equipped with a C18-bounded preparative RP-HPLC column (Phenomenex Kinetex 21.2 mm×150 mm, 5 μm). Peptides were analyzed by analytical HPLC (Shimadzu Prominance HPLC system) equipped with a C18-bounded analytical RP-HPLC column (Phenomenex Kinetex, 4.6 mm×150 mm, 5 μM) using a gradient elution (10-90% acetonitrile in water (0.1% TFA) over 20 min; flow rate=1.0 mL/min; diode array UV detector). Molecular weights of compounds were confirmed by HMRS-ESI-mass spectrometry using a Q Exactive Orbitrap LC-MS/MS (Termo Fisher Scientific Walthman, MA, USA).

The synthesis of the peptides was accomplished using a 2-Cl-TrtCl resin (58 mg, 0.09 mmol, 1.56 mmol/g), which was first swelled in anhydrous DMF over 30 min and then drained on solid phase peptide manifold without any further treatment. Then a solution of Fmoc-L-Pen(Trt)-OH (18 mg, 0.03 mmol, 0.33 equiv relative to the initial loading of the resin) and DIPEA (16 μL, 0.09 mmol, 1 equiv relative to the initial loading of the resin) in anhydrous DMF (1.5 mL) was added and the so obtained mixture was shaken overnight at room temperature. The residual chloride reactive groups were capped by adding a previously mixed solution of DIPEA (16 μL, 0.09 mmol, 1 equiv relative to the initial loading of the resin) in DCM/MeOH (9:1, 3 mL) and allowing the resin to gently shake for 30 min. The resin was washed with DMF (3×0.5 min), DCM (3×0.5 min) and Et₂O (3×0.5 min), dried exhaustively and its loading level was quantified (approx. 0.03 mmol/g) by measuring the level of the first amino acid attachment as already described in literature. The remaining cycles of peptide bond formation and AP-Fmoc removal reactions required to build the linear sequences were both performed placing the reactors in an ultrasonic bath (SONOREX RK 52 H by BANDELIN electronic, Germany). Specifically, after swelling again the resin in DCM/DMF 1:1, the Fmoc-protecting group was removed by treatment with a 20% piperidine solution in DMF (1×0.5 min and 1×1 min). The so obtained free amine was reacted with subsequent amino acid using 3 equivalents (relative to the 0.03 mmol/g loading) of Fmoc-protected amino acid and coupling reactants. Briefly, to a pre-stirred solution of Fmoc-L-His(Trt)-OH (56 mg, 0.09 mmol, 3 equiv), HBTU (35 mg, 0.09 mmo, 3 equiv) and HOBt (14 mg, 0.09 mmol, 3 equiv) in DMF (1.5 mL), DIPEA (32 μL, 0.18 mmol, 6 equiv) was added portionwise and the so obtained solution poured to the pre-swelled resin. The mixture was placed in the ultrasonic bath for 5 min at room temperature and then washed with DMF (3×1 min) and DCM (3×1 min). Subsequently, to cap the remaining free amines the resin was treated with 1.2 mL of an acetylating solution containing 2 equiv of Ac₂O (relative to the 0.03 mmol/g loading) and 4 equiv of DIPEA (relative to the 0.03 mmol/g loading) in DMF and then placed in the ultrasonic bath for 2 min. Elongation of the linear peptide sequence was obtained by iterative cycles of the aforementioned Fmoc deprotection and coupling reaction steps till 4-(Fmoc-aminomethyl)benzoic acid was anchored to the resin.

For the synthesis of reference Fmoc-AMBHA-R54, the peptide cleaved from the resin treating with a solution of TFA/TIS (95:5, 1.5 mL) for 1 h at room temperature. The resin was filtered and the crude peptide precipitated from the TFA solution diluting to 15 mL with cold Et₂O and then centrifuged (6000 rpm×15 min). The supernatant was carefully removed and the precipitate washed again with another volume of Et₂O as described above. The resulting wet solid was dried for 1 h under reduced pressure. An aliquot of the crude reduced peptide was dissolved in water/acetonitrile (9:1) and analyzed, before cyclization step, by reverse-phase analytical HPLC (solvent A: water+0.1% TFA; solvent B: acetonitrile+0.1% TFA; from 10 to 60% of solvent B over 30 min, flow rate: 1 mL min-1). Oxidation of sulfide side chains was achieved dissolving the crude peptide in H₂O (30 mL, approx. 0.5 mM) and adding an aqueous solution (5 mL) of N-chlorosuccinimide (5 mg, 0.036 mmol, 1.2 equiv) dropwise. The reaction was allowed to stir at room temperature for 30 minutes and complete conversion of the starting compound ascertained by HPLC profile comparison.

For the synthesis of DOTA- and NOTA-AMBHA-R54, the 4-(Fmoc-aminomethyl)benzoic acid functionalized resin underwent the last Fmoc deprotection and was functionalized with DOTA-tris(tBu)ester or NOTA-bis(tBu)ester using PyAOP and HOAt as coupling reactants. To a pre-stirred solution of DOTA-tris(tBu)ester (45 mg, 0.075 mmol, 2.5 equiv) (or NOTA-bis(tBu)ester: 31 mg, 0.075 mmol, 2.5 equiv), PyAOP (39 mg, 0.075 mmol, 2.5 equiv) and HOAt (10 mg, 0.075 mmol, 2.5 equiv) in DMF/DCM 2:1 (1.5 mL), DIPEA (26 μL, 0.15 mmol, 5 equiv) was added and the so obtained solution poured to the pre-swelled resin. The mixture was placed in the ultrasonic bath for 10 min at room temperature and the completion of the reaction ascertained by Kaiser ninhydrin and TNBS tests. The resin was washed with DMF (3×1 min), DCM (3×1 min), and Et₂O (3×1 min) and then dried exhaustively. The cleavage of the peptides from the resin and the subsequent oxidation was carried out as above describe for AMBHA-R54.

At this stage the reaction solutions of AMBHA-R54 and DOTA-and NOTA-R54 were freezed, lyophilized and the crude mixtures purified by reverse-phase preparative HPLC (solvent A: water+0.1% TFA; solvent B: acetonitrile+0.1% TFA; from 0 to 30% of solvent B over 25 min, flow rate: 10 mL min-1). Fractions of interest were evaporated from organic solvents under reduced pressure, freezed and then re-lyophilized. Obtained products were characterized by analytical HPLC and MS-ESI spectrometry. Yield, purity, retention times, and analytical data are reported as follows.

Fmoc-AMBHA-R54. 36 mg, overall yield: 83%, purity: 95%, tR 28.25 min, (analytical HPLC, 10 to 60% acetonitrile (0.1% TFA) in water (0.1% TFA) over 30 min, flow rate of 1.0 mL/min); HRMS (ESI-MS): Calculated: 1435,63180 for C₇₁H₈₉N₁₇O₁₂S₂ [M+H]+, found: 1436,64050

NOTA-AMBHA-R54. 29 mg, overall yield: 64%, purity: 95%, tR 16.25 min, (analytical HPLC, 10 to 60% acetonitrile (0.1% TFA) in water (0.1% TFA) over 30 min, flow rate of 1.0 mL/min); tR 23.48 min, (analytical HPLC, 0 to 30% acetonitrile (0.1% TFA) in water (0.1% TFA) over 30 min, flow rate of 1.0 mL/min); HRMS (ESI-MS): Calculated: 1499,70402 for C₆₈H₉₉N₂₀O₁₅S₂ [M+H]⁺, found: 1500,71305

DOTA-AMBHA-R54. 26 mg, overall yield: 56%, purity: 95%, tR 15.56 min, (analytical HPLC, 10 to 60% acetonitrile (0.1% TFA) in water (0.1% TFA) over 30 min, flow rate of 1.0 mL/min); tR 22.80 min, (analytical HPLC, 0 to 30% acetonitrile (0.1% TFA) in water (0.1% TFA) over 30 min, flow rate of 1.0 mL/min); HRMS (ESI-MS): Calculated: 1600,75170 for C₇₂H₁₀₆N₂₁O₁₇S₂ [M+H]⁺, found: 1601,76010

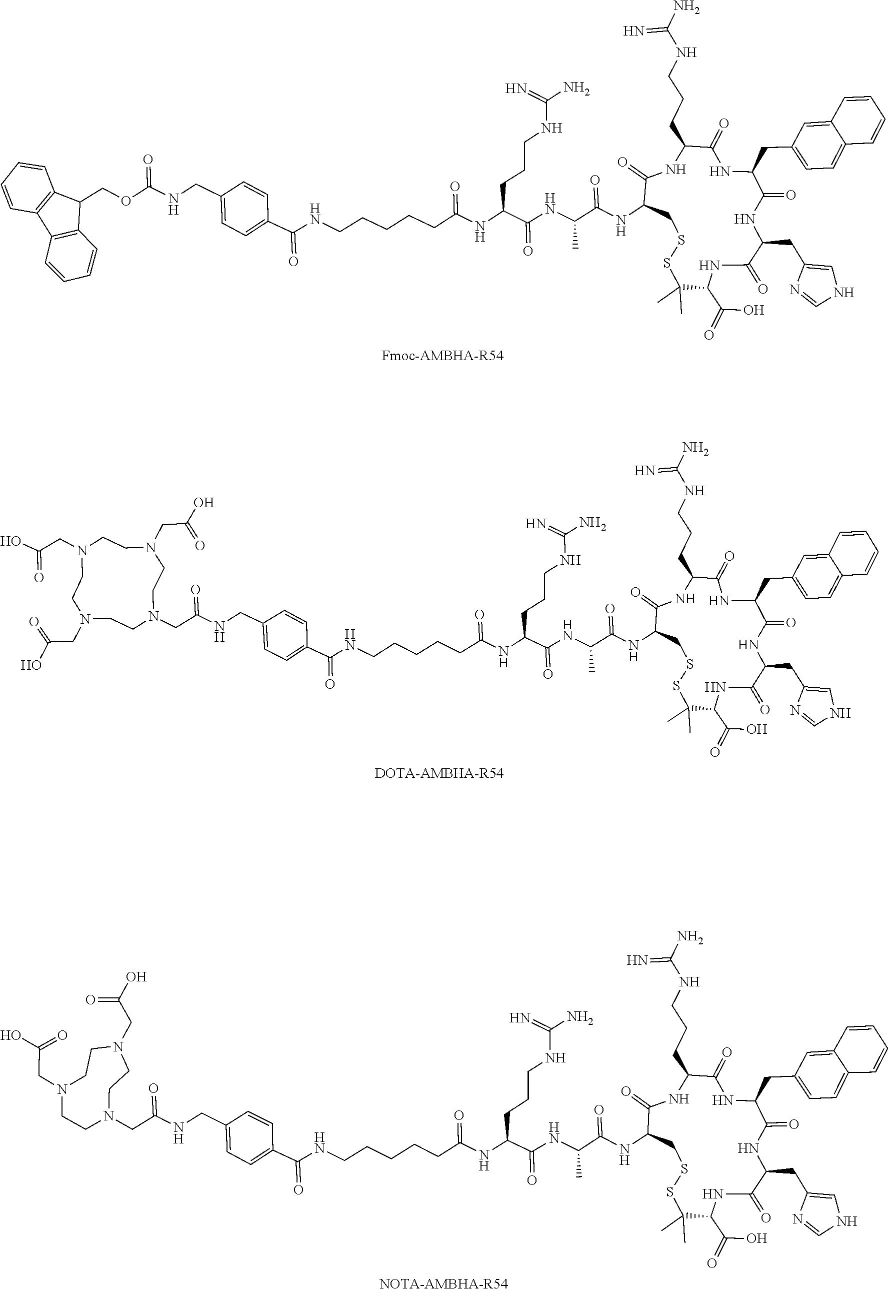

⁶⁸Ga-labeling of NOTA- and DOTA-AMBHA-R54 The non-processed fractionated eluate (1.6 mL containing 80% of the total eluted activity) of a ⁶⁸Ge/⁶⁸Ga-generator with TiO₂ matrix (GalliaPharma, Eckert&Ziegler; eluent: 0.1 M aq. HCl, total ⁶⁸Ga activity: 148-222 MBq) was adjusted to pH 2,5 by adding HEPES buffer (5 mL of a 0.1 M aq. solution) and used for labelling of 50 nmol of NOTA- or DOTA-Ahx-R54 for 10 minutes at 124° C. The labeled product was isolated by passing the reaction mixture through a C18 light solid phase extraction cartridge (SepPak), which was then purged with water (5 mL), followed by elution of the respective ⁶⁸Ga-labeled peptide using 0.4 mL pure ethanol. The product fraction was diluted with saline and sterilized by filtration through a 0.22 μm membrane filter. Radiochemical yields (before sterile filtration) were 80±5% based on ⁶⁸Ga-chloride starting activity. Radiochemical purity was determined via radio-TLC using silica coated TLC strips and 0.1 M Na-citrate pH 5 as mobile phase. Strips were analyzed using a Cyclone Plus storage phosphor system (PerkinElmer). Radiochemical purity was always >99%.

Cell Culture

Human T-cell Leukemia CCRF-CEM cells were obtained from the NCI 60 cancer cell line collection and grown in RPMI-1640 with 10% FBS and 2 mM glutamine and 100 units/mL of penicillin/streptomycin. Chinese hamster ovary (CHO) and CHO cells expressing human CXCR4 (CHO-hCXCR4) grown in complete medium supplemented with 1 mg/mL G418 (a kind gift of Dr. David McDermott (NIAID, NIH, Bethesda, Md.).

Flow Cytometry

CXCR4 levels were evaluated by flow cytometry using PE-conjugated anti-human CXCR4 (clone 12G5) (Catalog #FAB170, R&D Systems). Flow data were acquired using a FACSAria II Cell Sorter cytometer (BD Biosciences) and analyzed using FACS Diva 8 (BD Biosciences).

Determination of CXCR4 Affinity

Binding affinities to hCXCR4 were assessed with CCRF-CEM cells (1·10⁶ cells/sample) in binding buffer (PBS containing 50 mM HEPES, 1 mM CaCl₂, 5 mM MgCl₂, 0.5% BSA, and 0.3 mM NaN₃) and [¹²⁵I]CXCL12 (Perkin-Elmer, 2200 Ci/mmol) as radioligand. Cells were incubated with 100.000 cpm of the respective radioligand (approx. 0.1 nM) plus increasing concentrations (10⁻¹¹ to 10⁻⁵ M) of the respective peptide of interest (n=3 per concentration, total sample volume 250 μL). After incubation at 4° C. for 60 min (CCRF-CEM cells) at room temperature (RT), samples were centrifuged (5 min, 450 g, Megafuge 1.0, Heraeus Thermo Scientific), the supernatant was carefully removed and pooled with the supernatants of two subsequently performed washing steps. Then, the amount of free (pooled supernatants) and bound radioligand (cell pellet) was quantified using a γ-counter and expressed in percent of total added activity. The half maximal inhibitory concentration (IC₅₀) values were calculated using GraphPad Prism software (GraphPad Software, Inc., California).

Mouse Tumor Models

Female athymic nude mice (6 weeks old from Envigo Laboratories, Indianapolis, Ind., USA) were subcutaneously (s.c.) injected with 10⁷ CHO (right flank) and CHO-hCXCR4 (left flank) or CHO-hCXCR4 cells alone (right flank). Once tumors became palpable (100=³ [volume=0.5×long diameter×(short diameter)²]), approximately 15-21 days post injection (p.i.), the animals were used for the experiments.

Biodistribution Studies

10-15 MBq of the respective ⁶⁸Ga-labeled peptide in 100 μL of PBS were injected into the tail vein of nude mice bearing CHO-hCXCR4 tumor (groups of n=3-5). For competition studies, 8 mg/kg of unlabeled R54, were co-injected with the radioligands. At 45 min p.i., blood was collected from the retro-orbital plexus (100 μl) and mice were sacrificed and the tissues and organs of interest were dissected, weighed, and counted for radioactivity in a γ-counter. Biodistribution data are given as percent injected dose per gram tissue, % ID/g (means±SD).

PET/CT-Imaging

Tumor-bearing CHO/CHO-hCXCR4 mice were i.v. injected with 10-15 MBq of the respective radiotracer in 100 μL of PBS. At 45 min p.i., mice were anaesthetized with Avertin and images were acquired using a Discovery 600 PET/TC scanner (GE healthcare) in 3D mode with measured attenuation correction (1 bed position, 6 min scanning time). Data were decay-corrected to the time of tracer injection. The reconstructed images including PET, CT and fused PET/TC images, were generated on the advantage workstation version (AW4.6 software, GE healthcare). Tissue activity (e.g. tumor, lung, liver, kidney) was quantified considering the volume of a region of interest (ROI) to determine maximum standardized uptake values (SUVmax). Non-specific tracer accumulation was investigated by co-injection of 8 mg/kg unlabeled peptide R54.

Immunohistochemistry

CHO and CHO-hCXCR4 tumors were microscopically analyzed (H&E) and CXCR4 expression was evaluated through immunohistochemistry (IHC) (mab172, clone 44716, diluition 1:1000R&D system). The percentage of positive cells was evaluated in at least 5 areas (HPF 200× magnification) (Zeiss AxioScope light microscope).

Statistical Analysis

Statistical analysis was performed using the MedCalc 9.3.7.0 and Excel software. Unpaired Student t test was used for statistical analysis of ex vivo biodistribution and PET/TC images. The Spearman's correlation was used to measure the degree of association between SUV(max) and % CXCR4 IHC staining. All statistical tests were performed two-sided and a p-value <0.05 was considered to indicate statistical significance.

### RESULTS

CXCR4 Affinities

To assess the influence of modifications and chelator conjugation on R54, the CXCR4 affinity of Fmoc-AMBHA-R54 (see above the structure), NOTA-AMBHA-R54 and DOTA-AMBHA-R54 peptides as well as unmodified R54 were tested in competitive binding with [¹²⁵I]CXCL12 as radioligand on CXCR4-expressing CCRF-CEM cells. Binding affinity data, in particular hCXCR4 binding affinities (IC₅₀ in nM), are summarized in Table 1. Each experiment was performed in triplicate, and results are means ±SD from a minimum of three separate experiments.

TABLE 1

| Peptide | IC₅₀ (nM) |
|---|---|
| R54 | 20.0 ± 2.0 |
| Fmoc-AMBHA-R54 | 38.5 ± 3.5 |
| DOTA-AMBHA-R54 | 107.0 ± 39.6 |
| NOTA-AMBHA-R54 | 36.0 ± 11.5 |

Compared to R54, Fmoc-AMBHA-R54 and NOTA-AMBHA-R54 displayed slightly decreased CXCR4 affinity, whereas DOTA conjugation clearly reduced CXCR4 affinity. In vivo biodistribution studies

The biodistribution of [⁶⁸Ga]DOTA-AMBHA-R54 and [⁶⁸Ga]NOTA-AMBHA-R54 was investigated in CHO-hCXCR4 bearing CD1 mice (45 min p.i.). Table 2 shows the biodistribution of [⁶⁸Ga]DOTA-AMBHA-R54 and [⁶⁸Ga]NOTA-AMBHA-R54 in CHO hCXCR4 xenograft bearing nude mice at 45 min p.i. Data are given in % ID/g and are means±SD.

TABLE 2

| | [⁶⁸Ga]DOTA-AMBHA-R54 | | [⁶⁸Ga]NOTA-AMBHA-R54 | |
|---|---|---|---|---|
| | | competition | | competition |
| | tracer only | (+8 mg/kg R54) | tracer only | (+8 mg/kg R54) |
| organ | (n = 4) | (n = 3) | (n = 5) | (n = 4) |
| blood | 0.41 ± 0.08 | 0.37 ± 0.11 | 1.80 ± 1.02 | 1.10 ± 0.18 |
| lung | 0.43 ± 0.10 | 0.34 ± 0.10 | 1.41 ± 0.90 | 0.85 ± 0.11 |
| liver | 0.35 ± 0.09 | 0.26 ± 0.11 | 0.90 ± 0.40 | 0.56 ± 0.09 |
| spleen | 0.26 ± 0.05 | 0.14 ± 0.06 | 0.97 ± 0.40 | 0.70 ± 0.16 |
| digestive | 0.19 ± 0.03 | 0.20 ± 0.03 | 0.77 ± 0.33 | 0.56 ± 0.14 |
| system | | | | |
| kidneys | 1.79 ± 0.57 | 2.13 ± 0.9 | 5.83 ± 2.60 | 6.04 ± 3.20 |
| muscle | 0.24 ± 0.18 | 0.21 ± 0.06 | 0.41 ± 0.20 | 0.41 ± 0.28 |
| CHO-hCXCR4 | 1.63 ± 1.02 | 0.37 ± 0.17 | 4.44 ± 0.84 | 0.95 ± 0.31 |
| tumor | | | | |

Both [⁶⁸Ga]DOTA-AMBHA-R54 and [⁶⁸Ga]NOTA-AMBHA-R54 showed rapid background clearance and very low accumulation in all non-target tissues except the kidney. [⁶⁸Ga]NOTA-AMBHA-R54 displayed higher CXCR4-mediated tumor accumulation, nicely competed with cold R54, as compared to [⁶⁸Ga]DOTA-AMBHA-R54, consistent with the higher CXCR4 affinity of the NOTA analog.

Co-injection of an excess of unlabeled competitor (unlabeled R54) efficiently reduced the tracer uptake to background levels, indicating high CXCR4 specificity of tracer uptake in the tumor xenografts, with the [⁶⁸Ga]DOTA- and [⁶⁸Ga]NOTA-AMBHA-R54 accumulation in all other tissues remaining essentially unaffected by the competition conditions. High tumor/background ratios was observed for both analogs with values for [⁶⁸Ga]NOTA-AMBHA-R54 being substantially higher (FIG. 2).

In Vivo CXCR4 PET Imaging

Representative PET/CT images of [⁶⁸Ga]DOTA-AMBHA-R54 and [⁶⁸Ga]NOTA-AMBHA-R54 in CHO-hCXCR4 bearing CD1 mice is shown in FIG. 3. [⁶⁸Ga]DOTA-AMBHA-R54 and [⁶⁸Ga]NOTA-AMBHA-R54 displayed efficient CXCR4 targeting, with the NOTA-analog confirming superior performance with more efficient background clearance and higher accumulation in the CHO-hCXCR4 xenografts (FIG. 3iii) as compared to the DOTA-peptide (FIG. 3i). Interestingly, the differences in SUVₘₐₓ obtained for [⁶⁸Ga]NOTA-AMBHA-R54 and [⁶⁸Ga]DOTA-Ahx-R54 (19.6±2.00 vs 2.02±0.14 respectively, p=0.05) were even higher than anticipated from the biodistribution experiments.

Upon co-injection of unlabeled competitor (R54) tumor accumulation was reduced to background levels (FIG. 3ii and 3iv), highlighting once more the CXCR4-dependent tumor accumulation of [⁶⁸Ga]DOTA-AMBHA-R54 and [⁶⁸Ga]NOTA-AMBHA-R54 in xenograft models.

[⁶⁸Ga]NOTA-AMBHA-R54 Uptake (SUVmax) in the Tumors Correlates with CXCR4 Expression

To exemplarily correlate [⁶⁸Ga]NOTA-AMBHA-R54 uptake with the CXCR4 expression level in tumor tissue, immunohistochemical staining for CXCR4 was performed in CHO-hCXCR4 and CHO tumors. In FIG. 4A the mean percentage of CXCR4 positive cells was 56.6 for CHO-hCXCR4 vs 16.05 for CHO tumors, respectively. The Spearman test revealed a strong correlation between SUVmax of [⁶⁸Ga]NOTA-AMBHA-R54 PET and the CXCR4 expression in the respective tumor specimen as determined by IHC(R²=0.903, p=0.045, FIG. 4B).

### CONCLUSION

This study has conclusively demonstrated that the development of CXCR4 targeted PET tracers based on the well characterized, and structurally as well as physicochemical, optimized R54 peptide scaffold is feasible and successful. Of the two compounds in this study, [⁶⁸Ga]NOTA-AMBHA-R54 showed superior overall tracer characteristics as compared to DOTA counterpart, i.e. faster clearance from non-target tissues and higher accumulation in CXCR4-expressing tumor xenografts. Thus, [⁶⁸Ga]NOTA-AMBHA-R54 can be considered a suitable candidate for rapid translation into a clinical context.

### REFERENCES

1. Scala S. Molecular Pathways: Targeting the CXCR4-CXCL12 Axis—Untapped Potential in the Tumor Microenvironment. Clin Cancer Res. 2015 Oct 1;21(19):4278-85.https://doi.org/10.1158/1078-0432.CCR-14-0914.
2. Chatterjee S, Azad BB, Nimmagadda S. The intricate role of CXCR4 in cancer. Adv Cancer Res 2014; 124:31-82.https://doi.org/10.1016/B978-0-12-411638-2.00002-1.
3. Zlotnik A, Burkhardt AM, Homey B. Homeostatic chemokine receptors and organ-specific metastasis. Nat Rev Immunol. 2011 Aug 25;11(9):597-606. https://doi. org/10.1038/nri3049.
4. Teicher B A, Fricker S P. CXCL12 (SDF-1)/CXCR4 pathway in cancer. Clin Cancer Res. 2010 Jun 1;16(11):2927-31.https://doi.org/10.1158/1078-0432.CCR-09-2329.
5. D'Alterio C, Avallone A, Tatangelo F, Delrio P, Pecori B, Cella L,et al. A prognostic model comprising pT stage, N status, and the chemokine receptors CXCR4 and CXCR7 powerfully predicts outcome in neoadjuvant resistant rectal cancer patients. Int J Cancer. 2014 Jul. 15;135(2):379-90.
6. Wang L, Chen W, Gao L, Yang Q, Liu B, Wu Z, et al. High expression of CXCR4, CXCR7 and SDF-1 predicts poor survival in renal cell carcinoma. World J Surg Oncol. 2012 Oct 7; 10:212.
7. Neve Polimeno M, Ieranò C, D'Alterio C, Simona Losito N, Napolitano M, Portella L,et al. CXCR4 expression affects overall survival of HCC patients whereas CXCR7 expression does not. Cell Mol Immunol. 2015 July;12(4):474-82.
8. Peng S B, Zhang X, Paul D, Kays L M, Ye M, Vaillancourt P et al. Inhibition of CXCR4 by LY2624587, a Fully Humanized Anti-CXCR4 Antibody Induces Apoptosis of Hematologic Malignancies. PloS one, 2016. p. e0150585. https://doi.org/10.1371/journal.pone.0150585.
9. O'Boyle G, Swidenbank I, Marshall H, Barker CE, Armstrong J, White SA et al. Inhibition of CXCR4-CXCL12 chemotaxis in melanoma by AMD11070. Br J Cancer. 2013 Apr 30;108(8):1634-40. https://doi.org/10.1038/bjc.2013.124.
10. Grande F, Giancotti G, Ioele G, Occhiuzzi M A, Garofalo

A. An update on small molecules targeting CXCR4 as starting points for the development of anti-cancer therapeutics. Eur J Med Chem. 2017 Oct 20; 139:519-530.https://doi.org/10.1016/j.ejmech.2017.08.027.
11. Våbenø J, Haug BE, Rosenkilde M M. Progress toward rationally designed small-molecule peptide and peptidomimetic CXCR4 antagonists. Future Medicinal Chemistry 2015,7(10), 1261-1283. https://doi.org/10.41 55/fmc.15.64.
12. Peled A, Abraham M, Avivi I, Rowe J M, Beider K, Wald H et al. The high-affinity CXCR4 antagonist BKT140 is safe and induces a robust mobilization of human CD34+ cells in patients with multiple myeloma. Clin Cancer Res. 2014 Jan 15; 20(2):469-79. https://doi.org/10.1158/1078-0432.CCR-13-1302.
13. Karpova D, Brâuninger S, Wiercinska E, Krâmer A, Stock B, Graff J, Martin Het al. Mobilization of hematopoietic stem cells with the novel CXCR4 antagonist P0L6326 (balixafortide) in healthy volunteers-results of a dose escalation trial. J Transl Med. 2017 Jan 3;15(1):2. https://doi.org/10.1186/s12967-016-1107-2.
14. Pernas S, Martin M, Kaufman P A, Gil-Martin M, Gomez Pardo P, Lopez-Tarruella Set al. Balixafortide plus eribulin in HER2-negative metastatic breast cancer: a phase 1, single-arm, dose-escalation trial. Lancet Oncol. 2018 June;19(6):812-824. https://doi.org/10.1016/S1470-2045(18)30147-5.
15. De Clercq E. AMD3100/CXCR4 Inhibitor. Front Immunol. 2015 Jun 8; 6:276. https://doi.org/10.3389/fimmu.2015.00276.
16. Zboralski D, Hoehlig K, Eulberg D, Frômming A, Vater A. Increasing Tumor-Infiltrating T Cells through Inhibition of CXCL12 with NOX-A12 Synergizes with PD-1 Blockade. Cancer Immunol Res. 2017 November; 5(11):950-956.https://doi.org/10.1158/23266066.CIR-16-0303.
17. Chen Y, Ramjiawan RR, Reiberger T, Ng MR, Hato T, Huang Y et al. CXCR4 inhibition in tumor microenvironment facilitates anti-programmed death receptor-1 immunotherapy in sorafenib-treated hepatocellular carcinoma in mice. Hepatology. 2015 May; 61(5):1591-602. https://doi.org/10.1002/hep.27665.
18. Feig C, Jones J O, Kraman M, Wells R J, Deonarine A, Chan DS et al. Targeting CXCL12 from FAP-expressing carcinoma-associated fibroblasts synergizes with anti-PD-L1 immunotherapy in pancreatic cancer. Proc Natl Acad Sci USA. 2013 Dec 10;110(50):20212-7. https://doi.org/10.1073/pnas.1320318110.
19. Weiss ID, Jacobson O, Kiesewetter D O, Jacobus J P, Szajek L P, Chen X, Farber J M. Positron emission tomography imaging of tumors expressing the human chemokine receptor CXCR4 in mice with the use of 64Cu-AMD3100. Mol Imaging Biol. 2012 Feb;14(1):106-14. https://doi.org/10.1007/s11307-010-0466-y.
20. Wang Z, Zhang M, Wang L, Wang S, Kang F, Li G et al., Prospective Study of (68)Ga-NOTA-NFB: Radiation Dosimetry in Healthy Volunteers and First Application in Glioma Patients. Theranostics. 2015 Apr 28;5(8):882-9. https://doi.org/10.7150/thno.12303.
21. Gourni E, Demmer 0, Schottelius M, D'Alessandria C, Schulz S, Dijkgraaf I et al., PET of CXCR4 expression by a (68)Ga-labeled highly specific targeted contrast agent. J Nucl Med. 2011 November; 52(11):1803-10. https://doi.org/10.2967/jnumed.111. 098798.
22. Wester H J, Keller U, Schottelius M, Beer A, Philipp-Abbrederis K, Hoffmann F et al. Disclosing the CXCR4 expression in lymphoproliferative diseases by targeted molecular imaging. Theranostics 2015 Mar 1;5(6):618-30. https://doi.org/10.7150/thno.11251.
23. Vag T, Gerngross C, Herhaus P, Eiber M, Philipp-Abbrederis K, Graner F P et al. First Experience with Chemokine Receptor CXCR4-Targeted PET Imaging of Patients with Solid Cancers. J Nucl Med. 2016 May; 57(5):741-6. https://doi.org/10.2967/jnumed.115.1 61034.
24. Werner RA, Weich A, Higuchi T, Schmid JS, Schirbel A, Lassmann M et al. Imaging of Chemokine Receptor 4 Expression in Neuroendocrine Tumors-a Triple Tracer Comparative Approach. Theranostics. 2017 Apr 5;7(6):1489-1498. https://doi.org/10.7150/thno. 18754.
25. Watts A, Singh B, Basher R, Singh H, Bal A, Kapoor R et al. 68Ga-Pentixafor PET/CT demonstrating higher CXCR4 density in small cell lung carcinoma than in non-small cell variant. Eur J Nucl Med Mol Imaging. 2017 May; 44(5):909-910. https://doi.org/10.1007/s00259-017-3622-7.
26. Li X, Heber D, Leike T, Beitzke D, Lu X, Zhang X, Wei Y et al. [68Ga]Pentixafor-PET/MRI for the detection of Chemokine receptor 4 expression in atherosclerotic plaques. Eur J Nucl Med Mol Imaging. 2018 April; 45(4):558-566. https://doi.org/10.1007/s00259-017-3831-0.
27. Lapa C, Reiter T, Werner RA, Ertl G, Wester H J, Buck AK et al. [68Ga]Pentixafor-PET/CT for Imaging of Chemokine Receptor 4 Expression After Myocardial Infarction. JACC Cardiovasc Imaging. 2015 December; 8(12):1466-1468. https://doi.org/10.1016/j.jcmg.20 15.09.007.
28. Reiter T, Kircher M, Schirbel A, Werner RA, Kropf S, Ertl G et al. Imaging of C-X-C Motif Chemokine Receptor CXCR4 Expression After Myocardial Infarction With [68Ga]Pentixafor-PET/CT in Correlation With Cardiac MRI. JACC Cardiovasc Imaging. 2018 Oct;11(10):1541-1543. https://doi.org/10.1016/j.jcmg. 2018.01.001.
29. Bouter C, Meller B, Sahlmann C O, Staab W, Wester H J, Kropf S, Meller J. 68Ga-Pentixafor PET/CT Imaging of Chemokine Receptor CXCR4 in Chronic Infection of the Bone: First Insights. J Nucl Med. 2018 February;59(2):320-326. https://doi.org/10.2967/jnumed.117.193854.
30. Portella L, Vitale R, De Luca S, D'Alterio C, Ieranò C, Napolitano M et al. Preclinical development of a novel class of CXCR4 antagonist impairing solid tumors growth and metastases. PLoS One. 2013 Sep 13;8(9):e74548. https://doi.org/10.1371/journal.pone 0.0074548.
31. Di Maro S, Trotta A M, Brancaccio D, Di Leva F S, La Pietra V, Ieranò C et al. Exploring the N-Terminal Region of C-X-C Motif Chemokine 12 (CXCL12): Identification of Plasma-Stable Cyclic Peptides As Novel, Potent C-X-C Chemokine Receptor Type 4 (CXCR4) Antagonists. J Med Chem. 2016 Sep 22;59(18):8369-80. https://doi.org/10.1021/acs.jmedchem.6b00695.
32. Di Maro S, Di Leva F S, Trotta A M, Brancaccio D, Portella L, Aurilio M et al. Structure-Activity Relationships and Biological Characterization of a Novel, Potent, and Serum Stable C-X-C Chemokine Receptor Type 4 (CXCR4) Antagonist. J Med Chem. 2017 Dec 14;60(23):9641-9652. https://doi.org/10.1021/acs. jmedchem.7b01062.
33. Merlino F, Tomassi S, Yousif A M, Messere A, Marinelli L, Grieco P, Novellino E, Cosconati S, Di Maro S. Boosting Fmoc Solid-Phase Peptide Synthesis by Ultrasonication. Org Lett. 2019 Aug 16;21(16):6378-6382. https://doi.org/10.102¹/acs.orglett.9b02283.
34. Di Maro, S.; Pong, R. C.; Hsieh, J. T.; Ahn, J. M. Efficient solid-phase synthesis of FK228 analogues as potent antitumoral agents. J. Med. Chem. 2008;51(21), 6639-41

1. A radiopharmaceutical compound targeting CXCR4 receptor, said compound comprising:
a) a cyclic peptide, as monomer or multimer, having the following formula I:
Arg-Ala-[D-Cy s-Arg-X-Y-Z]—COOH
wherein
X is L-2-Nal or L-Phe;
Y is L-Phe or L-His;
Z is L-Cys or L-Pen;
b) a linker, said linker being bound to the N-terminus of said cyclic peptide;
c) a chelator, said chelator being bound to said linker; and
d) a radioisotope, said radioisotope being bound to said chelator.

2. The radiopharmaceutical compound according to claim 1, wherein the linker is a combination of at least two subunits A and B, wherein A is bound to said chelator, whereas B is bound to the N-terminus of said cyclic peptide, subunits A and B being connected to each other by amide bond or by a subunit C, preferably by amide bond,
wherein
subunit A is chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected to each other by amide bond; or (4-Ethynylphenyl)methanamine, (3-Ethynylphenyl)methanamine, 3-Ethynylaniline, 4-Ethynylaniline, 4-Pentyn-1-amine, But-3-yn-1-amine, propargylamine, 3-Azido-1-propanamine, 4-azido-1-butylamine, 5-azido-1-pentylamine, 6-azido-1-hexylamine, when subunits A and B are connected to each other by a subunit C;
subunit B is chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected to each other by amide bond; or
2-azidobenzoic acid, 3-azidobenzoic acid, 4-azidobenzoic acid, 2-azidomethyl benzoic acid, 3-azidomethylbenzoic acid; 4-azidomethylbenzoic acid, Azido-(PEG)n-COOH wherein n is from 2 to 10, 8-azidooctanoic acid, 7-azidoheptanoic acid, 6-azidohexanoic acid, 5-azidovaleric acid, 4-azidobutyric acid, 3-butynoic acid, 4-pentynoic acid and 6-hexynoic acid, when subunits A and B are connected to each other by a subunit C; and
subunit C is chosen from the group consisting of 1,4 substituted 1,2,3 triazole or 1,5 substituted 1,2,3 triazole linkers.

3. The radiopharmaceutical compound according to claim 1, wherein the chelator is chosen from the group consisting of 1,4,7-triazacyclononane-triacetic acid (NOTA), 1,4,7, 10-tetraazacyclododecane-1,4,7,10-tetraacetic acid (DOTA), diethylenetriaminepentaacetic acid (DTPA), {4-[2-(bis-carboxymethyl-amino)-ethyl]-7-carboxymethyl-[1,4, 7]triazonan-1-yl}-acetic acid (NETA), 1,4,8,11-tetraaza-cyclotetradecane 1,4,8,11-tetraacetic acid (TETA), p-SCN-Bn-NOTA(C-NOTA), NODASA, NODAGA, C-DOTA, DOTAGA, TRAP(Azide)₁, TRAP(Azide)₂, TRAP(Azide)₃.

4. The radiopharmaceutical compound according to claim 1, wherein the radioisotope is chosen from the group consisting of ⁶⁸Ga³⁺, ⁶⁷Ga³⁺, ⁶⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺, ²¹²Pb²⁺ or ¹⁸F⁻.

5. A precursor compound of the radiopharmaceutical compound as defined by claim 1, comprising
a) a cyclic peptide, as monomer or multimer, having the following formula I:
Arg-Ala-[D-Cys-Arg-X-Y-Z]—COOH
wherein
X is L-2-Nal or L-Phe;
Y is L-Phe or L-His;
Z is L-Cys or L-Pen:
b) a linker, said linker being bound to the N-terminus of said cyclic peptide; and
c) a chelator, said chelator being bound to said linker.

6. The precursor compound according to claim 5, wherein the linker is a combination of at least two subunits A and B, wherein A is bound to said chelator, whereas B is bound to the N-terminus of said cyclic peptide, subunits A and B being connected to each other by amide bond or by a subunit C,
wherein
subunit A is chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected each other by amide bond; or (4-Ethynylphenyl)methanamine, (3-Ethynylphenyl)methanamine, 3-Ethynyl aniline, 4-Ethynyl aniline, 4-Pentyn-1-amine, But-3-yn-1-amine, propargyl amine, 3-Azido-1-propanamine, 4-azido-1-butylamine, 5-azido-1-pentylamine, 6-azido-1-hexylamine, when subunits A and B are connected to each other by a subunit C;
subunit B is chosen from the group consisting of 2-aminobenzoic acid, 3-aminobenzoic acid, 4-aminobenzoic acid, 2-aminomethylbenzoic acid, 3-aminomethylbenzoic acid, 4-aminomethylbenzoic acid, amino-(PEG)n-COOH wherein n is from 2 to 10, 8-amino octanoic acid, 7-amino heptanoic acid, 6-aminohexanoic acid, 5-aminovaleric acid, 4-aminobutyric acid, 3-aminopropionic acid, when subunits A and B are connected to each other by amide bond; or
2-azidobenzoic acid, 3-azidobenzoic acid, 4-azidobenzoic acid, 2-azidomethyl benzoic acid, 3-azidomethylbenzoic acid; 4-azidomethylbenzoic acid, Azido-(PEG)n-COOH wherein n is from 2 to 10, 8-azidooctanoic acid, 7-azidoheptanoic acid, 6-azidohexanoic acid, 5-azidovaleric acid, 4-azidobutyric acid, 3-butynoic acid, 4-pentynoic acid and 6-hexynoic acid, when subunits A and B are connected to each other by a subunit C; preferably 6 aminohexanoic acid; and
subunit C is chosen from the group consisting of 1,4 substituted 1,2,3 triazole or 1,5 substituted 1,2,3 triazole linkers.

7. The precursor compound according to claim 5, wherein the chelator is chosen from the group consisting of 1,4,7-triazacyclononane-triacetic acid (NOTA), 1,4,7,10-tetraazacyclododecane-1,4,7,10-tetraacetic acid (DOTA),
diethylenetriaminepentaacetic acid (DTPA), {4-[2-(bis-carboxymethylamino)-ethyl]-7-carboxymethyl-[1,4, 7]triazonan-1-yl}-acetic acid (NETA), 1,4,8, 11-tetraazacyclotetradecane1,4, 8,11-tetraacetic acid (TETA), p-SCN-Bn-NOTA(C-NOTA), NODASA, NODAGA, C-DOTA, DOTAGA, TRAP(Azide)₁, TRAP(Azide)₂, TRAP(Azide)₃.

8. A radiopharmaceutical composition comprising the radiopharmaceutical compound as defined in claim 1, in association with one or more excipients and/or adjuvants.

9. A pharmaceutical composition comprising the precursor compound as defined in claim 5, in association with one or more excipients and/or adjuvants.

10.-15. (canceled)

16. A method for obtaining a radiopharmaceutical compound comprising:
a) radiolabeling a precursor compound as defined in claim 5 with a radioisotope chosen from the group consisting of ⁶⁸Ga³⁺, ⁶⁷Ga³⁺, ⁶⁴Cu²⁺, ⁴⁴Sc³⁺, ⁴⁷Sc³⁺, ¹¹¹In³⁺, ¹⁷⁷Lu³⁺, ⁸⁶Y³⁺, ⁹⁰Y³⁺, ²²⁵Ac³⁺, ²¹³Bi³⁺, ²¹²Pb²⁺ or ¹⁸F⁻.

17. A kit for the preparation of a radiopharmaceutical compound comprising:
a precursor compound as defined in claim 5.

18. A method for the treatment of CXCR4 expressing disorders comprising administering a cyclic peptide, monomer or multimer, or a pharmaceutical composition comprising said cyclic peptide wherein said cyclic peptide has the following formula: 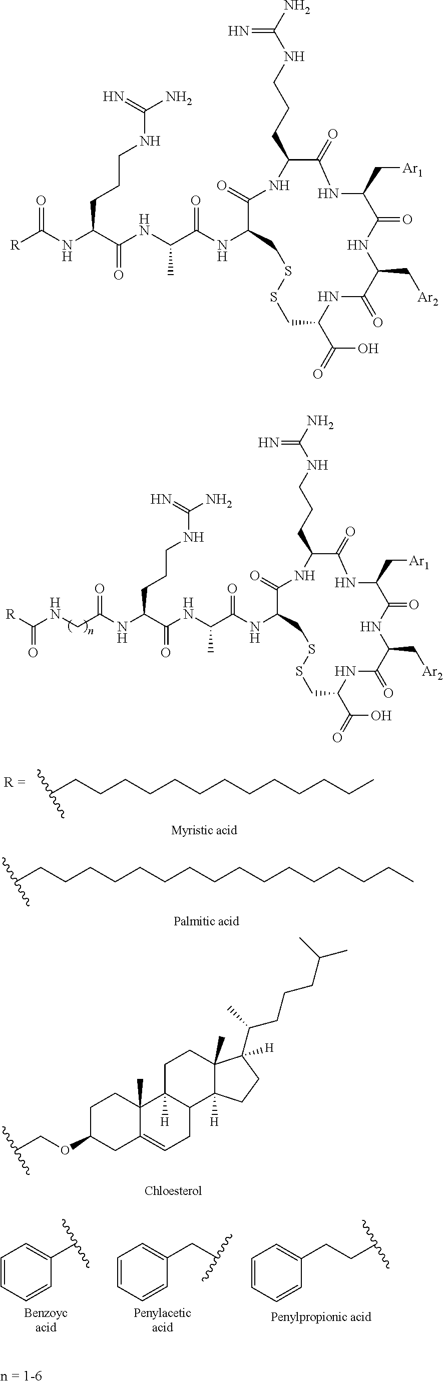 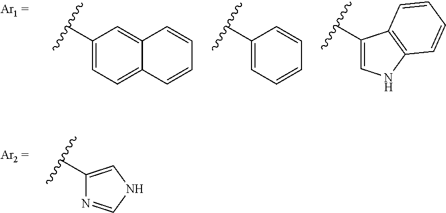
to a human in need of such treatment.

19. The method according to claim 18, wherein the CXCR4 expressing disorders are CXCR4 expressing tumours selected from the group consisting of Non-small-cell lung carcinoma, pancreatic cancer, prostate cancer, breast cancer, glioblastoma, sarcoma, colon cancer, melanoma, lung cancer, neuroendocrine tumors, and renal cancer.

20. The method according to claim 18 wherein said peptide is administered in association with an immunomodulating therapy.

21. A cyclic peptide or a pharmaceutical composition comprising said cyclic peptide, said cyclic peptide having formula II:
W-Arg-Ala-[D-Cys-Arg-X-Y-Z]—COOH
wherein
X is L-2-Nal or L-Phe;
Y is L-Phe or L-His;
Z is L-Cys or L-Pen, and
W is CH₃—(CH₂)ₙ—CO,
wherein n is from 1 to 30 when said cyclic peptide is chosen from the group consisting of:
(SEQ ID NO: 10)
CH₃(CH₂)ₙCO-Arg-Ala-[D-Cys-Arg-2-Nal-His-Pen]-COOH
(SEQ ID NO: 11)
CH₃(CH₂)ₙCO-Arg-Ala-[D-Cys-Arg-Phe-Phe-Cys]-COOH
(SEQ ID NO: 12)
CH₃(CH₂)ₙCO-Arg-Ala-[D-Cys-Arg-Phe-His-Pen]-COOH
(SEQ ID NO: 13)
CH₃(CH₂)ₙCO-Arg-Ala-[D-Cys-Arg-Phe-Phe-Pen]-COOH
and
(SEQ ID NO: 14)
CH₃(CH₂)ₙCO-Arg-Ala-[D-Cys-Arg-2-Nal-Phe-Pen]-
COOH;
and,
wherein n is from 0 to 30 when said cyclic peptide is chosen from the group consisting of:
(SEQ ID NO: 15)
CH₃(CH₂)ₙCO-Arg-Ala-[D-Cys-Arg-2-Nal-Phe-Cys]-COOH
(SEQ ID NO: 16)
CH₃(CH₂)ₙCO-Arg-Ala-[D-Cys-Arg-2-Nal-His-Cys]-COOH
and
(SEQ ID NO: 17)
CH₃(CH₂)ₙCO-Arg-Ala-[D-Cys-Arg-Phe-His-Cys]-COOH.

22. A method for the treatment of CXCR4 expressing disorders comprising administering a compound of claim 21 to a human in need of such treatment.

23. The method of claim 22, wherein the CXCR4 expressing disorders are selected from the group consisting of Non-small-cell lung carcinoma, pancreatic cancer, prostate cancer, breast cancer, glioblastoma, sarcoma, colon cancer, melanoma, lung cancer, neuroendocrine tumors, and renal cancer.

24. The method according to claim 22 wherein said peptide is administered in association with an immunomodulating therapy.
